(19) United States
(12) Patent Application Publication
(10) Pub. No.: US 2003/0175249 A1
(43) Pub. Date: Sep. 18, 2003

(54) Uses of the heat shock protein gp96

(76) Inventors: Harpreet Singh-Jasuja, Tubingen (DE); Hansjorg Schild, Rottenburg (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/338,115

(22) Filed: Jan. 7, 2003

Related U.S. Application Data
(63) Continuation of application No. PCT/EP01/07864, filed on Jul. 9, 2001.

(30) Foreign Application Priority Data
Jul. 10, 2000 (DE) 10033245.5

(51) Int. Cl. A61K 45/00; C12N 5/08

(52) U.S. Cl. 424/93.7; 435/372

(57) ABSTRACT

In a method for labeling or activating antigen-presenting cells (APCs) the APCs are contacted with gp molecules that do not carry interesting antigens.

The APCs are selected from the group consisting of dendritic cells, monocytes, macrophages, B cells and peritoneal exudate cells. Before the activation these APCs can be loaded with antigens and used in a method for inducing immune response or in tumor therapy.

### RELATED APPLICATION

This is a continuation application of international patent application PCT/EP01/07864, filed Jul. 9, 2001, designating the United States and published in German as WO 02/04516 A2, which claims priority to German application No. 100 33 245.5 filed Jul. 10, 2000.

### BACKGROUND OF THE INVENTION

1. Field of the Invention

The present invention relates to novel uses of the heat shock protein gp96.

Heat shock proteins are proteins whose expression is up-regulated during a heat shock or as response to stress. They act on the one hand as chaperones, and are thus involved in the protein folding process, and they also play a large part in the area of immunology in so-called antigen processing.

2. Related Prior Art

Conventional T lymphocytes depend on antigens being presented in the form of peptides. This function is assumed by antigen-presenting cells (APCs) which carry antigen-presenting molecules of the major histocompatibility complex (MHC) on their cell membrane. The antigen-representing cells (APCs) include, for example, dendritic cells, B cells (B lymphocytes) and macrophages, which are also referred to as professional APCs. In the interior of the APCs, protein antigens are digested by means of proteolytic enzymes to peptides which are then loaded onto the MHC molecules. Peptides for MHC class I are generated in particular in the cytoplasm and are bound to the MHC molecules in the endoplasmic reticulum. Peptides for MHC class II are produced by enzymatic digestion in lysosomes. The loaded MHC molecules are then transported to the cell surface. T cells recognize, with the aid of their membrane-associated antigen receptor, antigenic peptides together with these classical MHC molecules.

Only professional APCs are able to activate native T cells since they present antigens on MHC molecules and express costimulating molecules on the cell surface.

A distinction is made between the resting, immature APC which is specialized in antigen uptake, and the activated, mature APC which is specialized in antigen presentation. APCs can be activated by various reagents, e.g. by bacterial constituents such as lipopolysaccharide (LPS). Such constituents cause fevers in mammals, which is why they are also referred to as pyrogens. Other activators are, for example, cytokines such as TNF-alpha, which is in general use now in research for the activation of APCs.

It has been known for some time that the heat shock protein gp96 is able to mediate a tumor-specific immune response as long as it has been isolated from the tumor against which the response is to be directed. For example, a known tumor is induced in a mouse, this tumor is removed, and gp96 is isolated from the tumor. If this gp96 is then injected for immunotherapy into a mouse which is likewise affected by the same tumor, it is found that the tumor is rejected. For immunization on the other hand, gp96 is injected into a mouse and then the tumor is implanted or induced. It is found that the tumor is unable to grow. gp96 from non-tumor tissue cannot mediate this immune response.

The tumor specificity is accordingly not based on gp96 itself but on small peptides (antigens) which are associated on gp96. These peptides represent the information required by the immune system to recognize the tumor. In the current conceptual model, gp96 is taken up by the antigen-presenting cells (APCs).

Thus, inside the APCs the antigenic peptide bound to gp96 is transferred to MHC molecules, and the latter is presented on the surface in the context of the MHC molecules. This “crosspresentation” enables the antigenic peptide to be specifically recognized by T cells, which are activated thereby. Exogenous antigens include, for example, proteins, bacteria and apoptotic cells.

Accordingly, gp96 serves as an antigen carrier. A similar function has now also been shown for other heat shock proteins such as hsp70, hsp90, hsp110 and grp170. Since immunization with gp96 functions successfully even on use of extremely small amounts, there has already been speculation in the literature that APCs have specific receptors which bind on the surface, and endocytose, gp96 and other heat shock proteins.

Of all the hsps analyzed, the ER-internal heat shock protein gp96 has the best documented history in relation to the induction of specific CTL (cytotoxic T lymphocyte) responses.

The sequence for human gp96 is known for example from the publication by R. A. Mazzarella and M. Green (Erp99, an abundant, conserved glycoprotein of the endoplasmatic reticulum, is homologous to the 90-kDa heat shock protein (hsp90) and the 94 kDa glucose regulated protein (GRP94); J. Biol. Chem. 262: 8875-8883 (1987)), and the sequence for murine gp96 for example from the publication by R. G. Maki, L. J. Old and P. K. Srivastava (Human homologue of murine tumor rejection antigen gp96: 5′-regulatory and coding regions and relationship to stress-induced proteins; Proc. Natl. Acad. Sci. USA, 87: 5658-5662 (1990)).

WO 97/10002 discloses a method for the treatment or prevention of cancer and infectious diseases using sensitized APCs. For this purpose, the APCs are initially sensitized in vitro with a complex of a heat shock protein and an antigen molecule bound thereto, and then administered in an effective amount to the patient.

Singh-Jasuja et al., J. Exp. Med., 2000, 191: 1965-1974 were able to show that gp96 on the surface of APCs binds to one or more of yet unknown receptors, and this receptor-mediated uptake is essential for presentation of gp96-associated peptides on MHC class I molecules and thus for activation of T cells.

This publication discloses that gp96 binds to one or more of yet unknown receptors on human and murine APCs. A particularly important point in this connection is that so-called dendritic cells (DCs), which are reputed to have the greatest capacities as APC, bind gp96 well. The binding of gp96 to cells can be measured only if the protein is labeled in some form, e.g. radioactively, enzymatically or with a fluorescent dye. It is possible in the latter case to use gp96-FITC, that is to say gp96 which has been labeled with the fluorescent dye FITC (fluorescein isothiocyanate).

### SUMMARY OF THE INVENTION

In view of the above, it is an object of the present invention to provide at least one novel use for gp96.

The object is achieved according to the invention by using gp96 molecules, which carry no interesting antigenic peptides, for the labeling and/or activation of antigen-presenting cells (APCs).

“Interesting” antigenic peptides refer within the context of the present invention to peptides which are not presented by the antigen-presenting cells (APCs), or by which at least no immune response is induced. The gp96 molecules which carry no interesting antigenic peptides are referred to hereinafter as “unloaded” gp96 molecules.

This object is achieved according to the invention by making use of a gp96 function newly discovered by the inventors of the present application. This is because the inventors have found that not only is gp96 able to function as antigen carrier, on the contrary it is also, surprisingly, able when unloaded, i.e. not complexed with antigenic peptides, to activate APCs, in particular dendritic cells (DCs) and B cells, so that the latter in turn are even better able to activate T cells. This is because the inventors have found that on uptake of gp96 by the APCs, two processes take place simultaneously: firstly in this way gp96-associated antigen is efficiently taken up for presentation, but secondly the APC is also itself activated, after which its presentation ability is even better.

The inventors have found that unloaded gp96 activates human and murine APCs. This is shown by means of marker molecules which are up-regulated on the surface on activation of, for example, DCs: CD86 (B7.2), MHC class II and (only for human DCs) CD83. In addition, after activation with gp96, DCs secrete cytokines (FIG. 2b) which have important immunoregulatory functions. The cytokine IL-12 in particular is assuming an ever more important significance. Unloaded gp96 also activates murine B cells, which is shown by the greater expression of the surface molecules CD45R/B220, CD86 and MHC class II (H2-A^{b}).

Also significant is the demonstration that the activation is not based on contamination by pyrogens, e.g. LPS. The activation does not take place on denaturation of gp96 by heating, whereas LPS cannot be denatured by heating.

The therapeutic effect of activation of APCs with unloaded gp96, not loaded with antigens, consists of the fact that activated APCs are the only cells of the immune system able to activate náive T cells.

It is to be regarded as a crucial finding that resting APCs bind gp96 exceptionally well, but not APCs activated either by gp96 itself or LPS, which indicates that the receptor for gp96 on APCs is downregulated after activation thereof. The binding is measured for example through fluorescence-labeled gp96, that is to say gp96-FITC. It is thus possible to use gp96-FITC as detection reagent. The binding or nonbinding indicates the activation status of the APCs.

According to an object of the invention, unloaded gp96 molecules are used for the labeling and/or activation of antigen-presenting cells (APCs), the APCs preferably being selected from the group: dendritic cells (DC), monocytes, macrophages, B cells and peritoneal exudate cells.

It is moreover an object to use the gp96 molecules as markers for the activation and/or the maturation and/or the differentiation status of APC, in particular of DCs and B cells, and/or for immature DCs and B cells.

It is further preferred for the gp96 molecules to be labeled, preferably fluorescence labeled, further preferably FITC-labeled.

It is further preferred for the gp96 molecules to be obtained from primary nonhuman mammalian cells, preferably from mouse cells, or from human or murine cell lines, or else recombinantly in Escherichia coli or insect cells.

It is advantageous in this connection that gp96 molecules can be prepared firstly in any amount and secondly in such a way that they are not associated or are associated only with irrelevant antigens which are expressed, for example, in genetically modified mice, other mammals or else in Escherichia coli or insects.

It is moreover an object for the gp96 molecules being used for activating the maturation of APCs, in particular DCs or B cells.

The invention has as a further object a method for the in vivo or in vitro activation of APCs, in particular DCs or B cells, in which gp96 molecules are preferably used as described above. The gp96 molecules can in this case be injected for example subcutaneously or intradermally.

It is another object in this connection that the APCs are being loaded before the activation ex vivo with antigens which are preferably selected from the group: tumor-associated, tumor-specific, autoimmune-associated, viral and bacterial antigens.

It is further preferred moreover for the APCs to be treated in vitro with gp96 molecules alone or together with other factors such as TNF-alpha.

The invention has as a further object APCs prepared by the novel method, and the use thereof for inducing an immune response against the antigens with which they have been loaded ex vivo.

The invention has as another object the use of gp96 molecules for inducing tolerance and/or a TH2-type response and/or a TH1-type response against antigens, preferably against antigens which are selected from the group: tumor-associated, tumor-specific, autoimmune-associated, viral and bacterial antigens.

It is preferred to use nonactivated APCs with strongly expressed gp96 receptor which is preferably detected via labeled, preferably fluorescence-labeled, gp96 molecules, and to arrest such APCs in this nonactivated state by substances such as, for example, cytocahlsin D.

The invention as another object relates to the use of the novel APCs for tumor therapy and/or prevention, to a therapeutic composition with these APCs and a therapeutically acceptable carrier, and to a kit with gp96 molecules to be used according to the invention, and the necessary reagents.

Thus, gp96 is used according to the invention not as antigen carrier but as activator of APCs, in particular DCs and B cells. The APCs can then be loaded ex vivo with the desired antigen and, after the activation by gp96, injected back into the patient for therapy.

If gp96-FITC is employed as detecting agent in order to find the state of activation or differentiation of antigen-presenting cells, these APCs can be divided, according to their binding of gp96-FITC and thus according to their level of expression of the gp96 receptor, into various categories which are relevant for tumor therapy.

The invention further has as another object a method for the in vitro preparation of DCs from monocytes isolated from blood and/or stem cells prepared from bone marrow, in which the monocytes and/or stem cells are treated with gp96 molecules alone or in combination with growth factors such as, for example, GM-CSF.

This derives from the inventor's realization that gp96 molecules are able to differentiate such precursor cells to DCs.

In view of the above, the present inventors have tested the effect of unloaded gp96 on DC maturation and T-cell activation and were surprisingly able to show that immature DCs treated with gp96 secrete TNF-alpha and IL-12 and convert to the mature phenotype, and, in the case of human DCs, they express increased levels of CD86, MHC class II and CD83 molecules. This change in phenotype has functional consequences which are revealed for example by increase in the activation of allogeneic T cells.

It is of interest that, after maturation, the DCs lose their capacity to bind exogenous gp96. The gp96 receptor on mature DCs is downregulated, which suggests that this receptor behaves in a way similar to other receptors involved in antigen uptake, such as the scavenger receptor CD36, the mannose receptor or the integrins αᵥβ₃ and αᵥβ₅. This observation is in good agreement with the reduced ability of mature DCs to take up antigen.

The inventors show further that unloaded gp96 is able to activate B cells, which is demonstrated by the stronger expression of the surface molecules CD45R/B220, CD86 and MHC class II (H2-A^{b}).

### BRIEF DESCRIPTION OF THE DRAWINGS

Examples of results are shown as follows in the Figures:

FIG. 1: gp96 activates human dendritic cells.

Human dendritic cells were prepared in vitro from CD14+ PBMCs with GM-CSF and IL-4 after 7 days, and incubated with gp96, heat-treated gp96, LPS or heat-treated LPS for 24 h. A: CD86 expression levels of DCs treated with gp96/LPS (filled histogram), or untreated DCs (in gray; the black line represents an isotypic control antibody which showed the same fluorescence intensity for all treatments). B shows the percentage of high CD86 (as indicated in A by the marker bar) and activation marker CD83 expressing DCs after treatment with the different effector molecules. Error bars indicate the standard deviation. The results represent three independent experiments.

FIG. 2: gp96 activates mouse dendritic cells.

Mouse dendritic cells were prepared from bone marrow of C57BL/6 or BALB/c mice with GM-CSF after 7 days. A: treatment with 30 and 100 μg/ml gp96 and 2 μg/ml LPS and heat-treated LPS after 24 h led to up-regulation of CD86 (measured by FACS double staining with CD11c and CD86 antibodies), while heat-treated gp96 did not activate the DCs. B: supernatants from the above experiment were analyzed by ELISA for the content of the cytokines IL-12 and TNF-alpha (which showed similar results to those in A). The error bars show the standard deviation of three experiments in each case. The results represent at least three independent experiments.

FIG. 3: Human and mouse dendritic cells activated by gp96 are able to induce strong proliferation of alloreactive T cells.

Human DCs were prepared and treated for 24 h as described above with 30 μg/ml gp96, heat-treated gp96 or 2 μg/ml LPS. After extensive washing, these pretreated DCs were incubated with 1×10E5 PBMCs from another donor for 4 days in different stimulator/responder ratios (the ratio 1:30 is shown). The proliferation of T cells was assayed by adding 1 μCi of 3H-thymidine for 16 h. Error bars indicate the standard deviation of three experiments in each case. The results represent two independent experiments. Similar results were obtained with BALB/c mouse DCs, which induced a proliferation of C57BL/6 T cells (data not shown).

FIG. 4: Activated DCs downregulate the gp96 receptor.

DCs derived from bone marrow from C57BL/6 mice show a heterogeneous population of activated (high CD86 positive on FL-2) and nonactivated (low CD86 positive) CD11c-positive DCs. OVA-FITC as control protein did not bind at all, whereas gp96-FITC bound only to nonactivated DCs (lower panels). The upper panels show the CD11c expression of the DC preparation and the binding of gp96-FITC. The values indicate percentages of total cells in the defined quadrants. The results represent three different experiments.

FIG. 5: gp96 activates mouse B cells

Primary B cells were isolated from spleen cells from C57BL/6 mice. Treatment with 50 μg/ml gp96 led to up-regulation of CD86 (gray bars) and MHC class II (H2-A^{b}) molecules (black bars). The activation is comparable to that with LPS. Heat-inactivated gp96 shows no activation, like the negative control (medium), and it is thus possible to preclude LPS contamination in the gp96 preparation being responsible for the activation. The diagram shows the percentage of activated B cells labeled by CD 45R/B220 in combination with CD86 or MHC class II (H2-A^{b}).

### DESCRIPTION OF PREFERRED EMBODIMENTS

### EXAMPLE I

### Materials and Methods

Generation of Dendritic Cells

The medium used throughout was Iscove's modified Dulbecco's medium (IMDM, BioWhittaker, Verviers, Belgium) which was supplemented with 2 mM L-glutamine (GibcoBRL Life Technologies, Paisley, UK), 100 IU/ml penicillin/streptomycin (Gibco), 10% FCS (PAA, Linz, Austria) and cytokines as indicated below. Mouse immature DCs were generated from bone marrow of C57/BL/6 or BALB/c mice by the method of Inaba et al., J. Exp. Med. 1992, 176: 1693-1702. Briefly stated, bone marrow cells were incubated with 150 U/ml GM-CSF (PeproTech, London, UK) for 6-8 days, renewing the medium every two days. Approximately 90-100% of all the cells in the FACS(r) gate used for monocytes were DCs, as determined by flow cytometry with antibodies (which were obtained from Pharmingen, San Diego, Calif.): these were CD11c-, CD86- and MHC class II-positive and CD14-negative. Human immature dendritic cells were prepared from PBMCs by the method of Bender et al., J. Immunol. Methods 1996, 196: 121-135.

Briefly stated, CD14+ monocytes were purified by 1 h adherence to culture dishes and extensive washing. The monocytes were incubated with 1 000 IU/ml IL-4 and 800 IU/ml GM-CSF for 6-8 days, renewing the cytokines every three days. The cells generated in this way showed a large number of dendrites up to day 12 and were only slightly adherent. They expressed CD1a, low CD14, CD86, HLA-DR and very low CD83 on their surface, as was determined by antibodies (Pharmingen).

Preparation of Unloaded gp96 Molecules

Gp96 was purified from a mycoplasma-free IGELa2 mouse cell line as described in Arnold et al., J. Exp. Med. 1995, 182: 885-889. FPLC fractions which preceded and followed the fractions which contained no gp96 according to Western blot are referred to as “flanking fractions” (provided by Immatics Bio-technologies, Tübingen). Endotoxin which might be present in the gp96 preparations was tested by a Limulus Amebocyte Lysate Kit (QCL-1000, BioWhittaker) in accordance with the guidelines published by the US Food and Drug Administration (FDA). The endotoxin content was determined in all cases to be at or below 0.05 EU/μg of gp96. Possible mycoplasma contamination of the IGELa2 cell line and of the gp96 FPLC fractions was detected using the Mycoplasma Plus kit from Stratagene, La Jolla, Calif.

In case the gp96 molecules prepared in this way are still complexed with peptides, it is possible to cleave the antigenic peptides or components from the endogenous heat shock protein complexes by ATP incubation or by incubation in a buffer of low pH. Thus, for this purpose, the heat shock protein-peptide complex is either incubated with 10 mM ATP and 3 mM MgCl₂ at 37° C. for 1 h, or else the complex is exposed to 0.2% strength trifluoroacetic acid (TFA) at 4° C. for 1 hour in order to cleave the proteins. The samples are then put onto a Centricon 10 filter column with an exclusion membrane (Millipore) and centrifuged in order thus to separate the peptides from the heat shock proteins (see T. Ishiii et al., Isolation of MHC Class I-Restricted Tumor Antigen Peptide and its Precursors Associated with Heat Shock Protein hsp 70, hsp90, gp96,; J. Immunol., 162: 1303-1309 (1999)).

Stimulation of the Dendritic Cells

Mouse and human DCs were stimulated by addition of 30 to 100 μg/ml gp96 or 2 μg/ml LPS (from Salmonella typhimurium, Sigma Chemicals, St. Louis, Mo.) for 24 h. In some cases, gp96 or LPS were pretreated at 95° C. for at least 20 min.

Cytokine Detection

Mouse IL-12 (p40) and TNF-alpha were measured in culture supernatants using standard sandwich ELISA protocols. Antibodies and recombinant standards of both cytokines were obtained from Pharmingen. The capture antibody was bound to the ELISA plate (MaxiSorb™ Nunc, Roskilde, Denmark), and the biotinylated detection antibody was recognized by streptavidin-conjugated horseradish peroxidase and detected by an ABTS substrate (Sigma) which emitted at 415 nm.

Stimulation of Alloreactive T Cells

Human or BALB/c DCs were stimulated in a plate with 96 wells as described above, washed extensively and incubated with PBL from a different donor or C57BL/6 spleen cells, respectively, for 4 days with at different responder/stimulator ratios. On day 4, 1 μCi of 3H-thymidine was added to each well, the cells were harvested after a further 16 h, and the incorporated 3H-thymidine was detected by using a Wallac 1450 MicroBeta counter.

FACS Analysis

The cell surface was stained using antibodies as described above, ovalbumin-FITC or gp96-FITC (kindly provided by Immatics Biotechnologies, Tubingen), which were incubated with the cells for 30 min on ice in IMDM which contains 10% FCS. Dead cells were excluded by PI staining. All FACS(r) analyses were carried out in a FACSCalibur(r) (Becton Dickinson, Mountain View, Calif.), using a Cell Quest software.

### EXAMPLE 2

### Unloaded gp96 Induces the Maturation of Human DCs

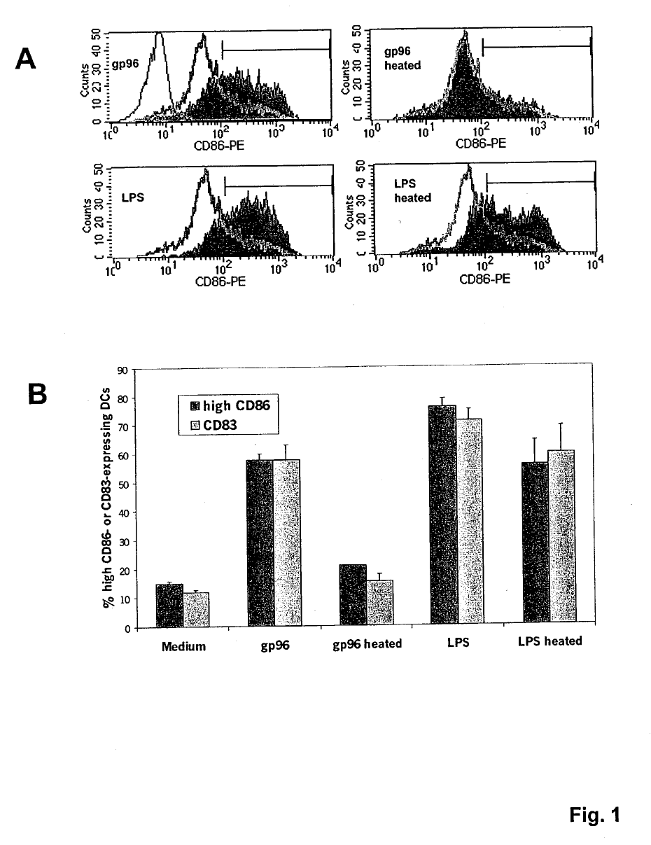

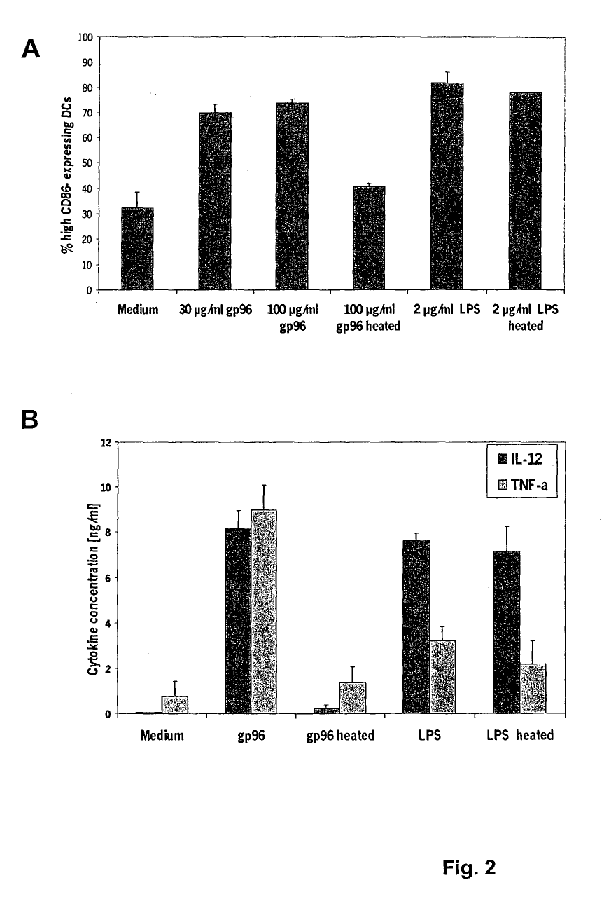

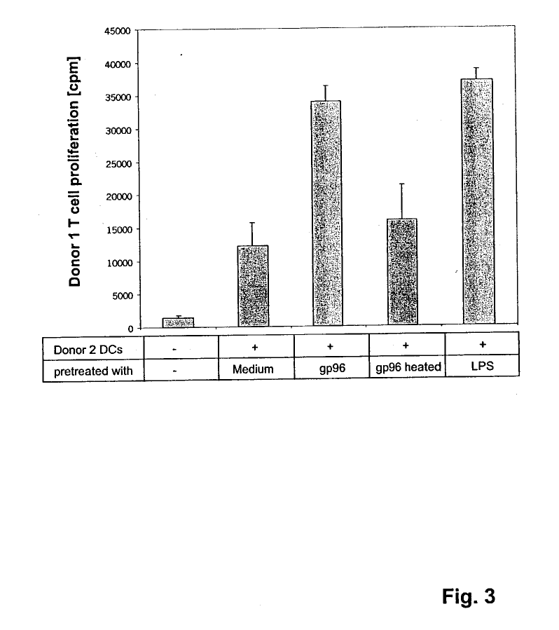

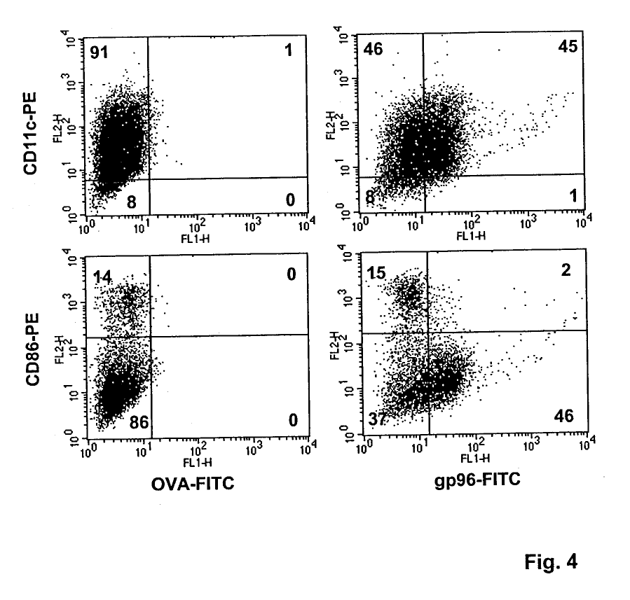

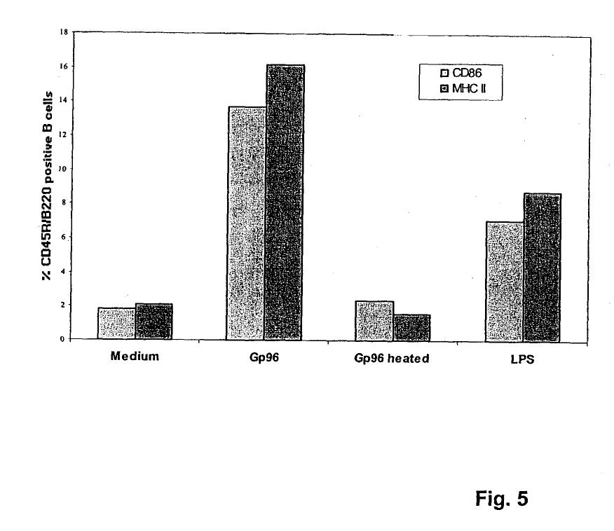

In order to study the effect of unloaded gp96 on phenotypical changes of DCs, immature DCs were generated by incubating human PBMCs with GM-CSF and IL-4 for 7 days. For a further 24 h, gp96 or LPS were added as a positive control to the cultures. As shown in FIG. 1, both gp96 and LPS induced maturation of the DCs, which then showed increased levels of CD83 and CD86 on their surface. Denaturation of gp96 by heat destroyed its ability to activate DCs, whereas LPS was unaffected by this treatment. These latter observations are a strong argument in favor of gp96-mediated DC activation not being a consequence of endotoxin contamination but the result of binding of native gp96 to its receptor. This was further supported by the finding that the gp96-flanking fractions from the FPLC purification (which lacked gp96) did not induce DC maturation and that normal medium and gp96 did not differ in their endotoxin content, as was measured by the Limulus Amebocyte Test Kit (data not shown). In addition, gp96 was purified from a cell line which was not infected with mycoplasma. It is important to state this, since it was recently shown that the supernatant of mycoplasma-infected cells is able to induce DC maturation. BSA and concanavalin A, which were added as control proteins in similar amounts as gp96, did not activate DCs (data not shown).

### EXAMPLE 3

### Unloaded gp96 Induces the Maturation of Mouse DCs

A comparable gp96-mediated activation was likewise obtained by using DCs derived from the bone marrow of mice. Incubation of immature DCs with unloaded gp96 at various concentrations induced a heat-labile maturation of DCs, as was demonstrated by an increased expression of CD86 molecules (FIG. 2A) and MHC class II molecules (data not shown). In addition to the expression of maturation markers on the cell surface, gp96 likewise induces secretion of the inflammation-promoting cytokines IL-12 and TNF-alpha (FIG. 2B). The effect is once again heat-sensitive and is not based on endotoxin contamination possibly present in our gp96 preparations.

### EXAMPLE 4

### Gp96-Activated DCs Induce Strong T-Cell Proliferation

In order to investigate whether gp96-mediated DC maturation has functional consequences, DCs caused to mature by gp96 or LPS were incubated with allogeneic PBMCs for 4 days, and the cell proliferation was determined by incubation with 3H-thymidine. As shown in FIG. 3, DCs which showed a mature phenotype either after LPS or gp96 activation for 24 h induced T-cell proliferation 3 times better than did immature DCs which were incubated only with medium. As already observed previously, the gp96-mediated effect is heat-sensitive, because DCs incubated with heated gp96 showed no increase in the capacity to stimulate T-cells. A comparable T-cell proliferation was observed on use of mouse DCs and allogeneic spleen cells (data not shown)

### EXAMPLE 5

### Mature DCs Express Reduced Levels of the gp96 Receptor

Maturation of DCs induces up-regulation of MHC class II, CD83 and CD86 molecules, leading to increased T-cell proliferation. In addition, when once activated, DCs are no longer able to receive gp96-mediated stimuli. As shown in FIG. 4, all CDllc-positive mouse DCs bind gp96, but not ovalbumin. However, only immature DCs which express low levels of CD86 are able to bind gp96. Since gp96 is complexed with peptides from the cell from which it has been isolated, and DCs are capable of cross-presentation of these peptides on MHC class I molecules, it can no longer be expected that mature DCs will be able to present gp96-associated peptides for T cells.

### EXAMPLE 6

### Unloaded gp96 Activates Primary Mouse B Cells

Primary B cells were isolated from spleen cells from C57BL/6 mice by negative magnetic depletion (MACS™, Miltenyl Biotech) using an antibody against CD43 which had been conjugated to superparamagnetic microbeads (Miltenyl Biotech). CD43 is expressed by all spleen cells apart from resting peripheral B cells. The B cells are activated by adding 50 μg/ml gp96 to 300 000 B cells. Untreated B cells (“medium”) serve as negative controls, and 2 μg/ml lipopolysaccharide (LPS, Sigma-Aldrich) as positive control. In order to preclude the possibility of contamination, heat-inactivated gp96 (20 min at 95° C., “gp96 boiled”) is also tested. Evaluation takes place alternatively after 2 or 3 days in flow cytometry (FACSCalibur, BectonDickinson) by measuring the surface molecules CD45R/B220, CD86 and MHC class II with the aid of fluorescence-labeled antibodies (Becton-Dickinson Pharmingen). As FIG. 5 shows, primary mouse B cells are activated by unloaded gp96. This is shown by the stronger expression of the surface molecules CD45R/B220, CD86 and MHC class II (H2-A^{b}) compared with the expression of these antigens in cells treated only with medium (“medium”). Heat-inactivated gp96 (“gp96 boiled”) shows, just as with medium, no activating effect, which makes it possible to preclude LPS contamination in the gp96 preparation being responsible for the observations.

### EXAMPLE 7

### Conclusion

The above experiments show that the ER-internal heat shock protein gp96 is able, even unloaded, to induce maturation of mouse and human DCs and of B cells. This observation is particularly remarkable in the light of the earlier findings showing that gp96 binds specifically to DCs, which led to a cross-presentation of gp96-associated peptides restricted to MHC class I. The finding of downregulation of the gp96 receptor on the surface of mature DCs additionally permits initial speculations about the nature of the gp96 receptor which is expressed on DCs. Possible candidates are endocytic receptors such as the scavenger receptor CD36 or the integrins αvβ3 and αvβ5, all of which have been shown to be downregulated in DC maturation.

The invention provides the first evidence that gp96 is not only a peptide carrier which directs associated peptides to professional APCs, but also a direct activator of DCs and B cells. Gp96 might therefore act as a danger signal when it is released from necrotic or stressed cells which deliver both unspecific and specific stimuli to the immune system.

Once they have been activated, DCs lose the capacity to take up new gp96-complexed peptides, and gain the ability to communicate efficiently with T cells which are specific for the presented MHC-peptide complexes. This situation greatly resembles that observed initially for the presentation of soluble antigens on MHC class II molecules, where it was described that DCs are “arrested” in a state of antigen presentation and are highly efficient at activating T cells.

This novel feature of gp96 provides an additional, previously unknown, explanation of its high immunogenicity and will permit improvement in its use for the induction of specific immune responses in vivo.

What is claimed is:

1. A method for treating antigen presenting cells (APCs), comprising
contacting the APCs with unloaded gp96 molecules, and
labeling said antigen presenting cells with the unloaded gp96 molecules.

2. The method of claim 1, wherein the APCs are selected from the group consisting of: dendritic cells (DCs), monocytes, macrophages, B cells and peritoneal exudate cells.

3. The method of claim 1, wherein the gp96 molecules function as a marker for a status of the APCs, said status being selected from the group consisting of the activation, maturation and differentiation.

4. The method of claim 3, wherein the APCs are selected from the group consisting of DCs, B cells, immature DCs and immature B cells.

5. The method of claim 1, wherein the gp96 molecules are fluorescent-labeled.

6. The method of claim 5, wherein the gp96 molecules are FITC-labeled.

7. The method of claim 1, wherein the gp96 molecules are obtained from the group consisting of mammalian cell lines, human cell lines, insect cell lines, primary, nonhuman, mammalian cells, murine cells, recombinantly from Escherichia coli, and recombinantly from insect cells.

8. A method for treating antigen presenting cells (APCs), comprising the steps of:
contacting the APCs with unloaded gp96 molecules, and
activating antigen presenting cells.

9. The method of claim 8, wherein the APCs are selected from the group consisting of: dendritic cells (DCs), monocytes, macrophages, B cells and peritoneal exudate cells.

10. The method of claim 8, wherein the gp96 molecules are fluorescent-labeled.

11. The method of claim 8, wherein the gp96 molecules are FITC-labeled.

12. The method of claim 8, wherein the gp96 molecules are obtained from the group consisting of mammalian cell lines, human cell lines, insect cell lines, primary, nonhuman, mammalian cells, murine cells, recombinantly from Escherichia coli, and recombinantly from insect cells.

13. The method of claim 8, further comprising activating maturation of APCs.

14. The method of claim 8, wherein said steps are performed in vitro.

15. The method of claim 8, wherein said steps are performed in vivo.

16. The method of claim 15, further comprising the step of injecting of the unloaded gp96 molecules into a human individual.

17. The method of claim 1, further comprising loading the APCs with antigens before contacting the APCs with unloaded gp96 molecules.

18. The method of claim 14, further comprising loading the APCs with antigens before contacting the APCs with unloaded gp96 molecules.

19. The method of claim 15, further comprising loading the APCs with antigens before contacting the APCs with unloaded gp96 molecules.

20. The method of claim 17, wherein the antigens are selected from the group consisting of tumor-associated, tumor-specific, autoimmune-associated, viral and bacterial antigens.

21. The method of claim 18, wherein the antigens are selected from the group consisting of tumor-associated, tumor-specific, autoimmune-associated, viral and bacterial antigens.

22. The method of claim 19, wherein the antigens are selected from the group consisting of tumor-associated, tumor-specific, autoimmune-associated, viral and bacterial antigens.

23. The method of claim 14, wherein the APCs are treated in vitro with gp96 molecules and additionally with other factors.

24. The method of claim 23, wherein the other factors comprise TNF-alpha.

25. The method of claim 17, further comprising inducing the immune response against the antigens.

26. The method of claim 18, further comprising inducing the immune response against the antigens.

27. The method of claim 19, further comprising inducing the immune response against the antigens.

28. The method of claim 20, further comprising inducing tolerance and/or TH2-type response and/or TH1-type response against said antigens.

29. The method of claim 28, further comprising adding fixing substances for arresting the APCs in the nonactivated state, wherein the nonactivated APCs have high expression of a gp96 receptor.

30. The method of claim 29, wherein the fixing substances comprise Cytochalasin D.

31. The method of claim 30, further comprising detecting the gp96 receptor via labeled gp96 molecules.

32. A method of treatment, comprising
activating APCs according to a method selected from the group consisting of the methods of any of claims 1, 8, 34 or 38, and
administering the activated APCs to a human being, thereby treating a tumor.

33. A method of treatment, comprising
activating APCs according to a method selected from the group consisting of the methods of any of claims 1, 8, 34 or 38, and
administering the activated APCs to a human being, thereby preventing a tumor.

34. A method for in vitro preparation of dendritic cells from monocytes isolated from blood, comprising treating the monocytes with gp96 molecules.

35. The method of claim 34, wherein the gp96 molecules are in combination with growth factors.

36. The method of claim 35, wherein the growth factor is GM-CSF.

37. The method of claim 34, wherein the gp96 molecules are unloaded gp96 molecules.

38. A method for in vitro preparation of dendritic cells from stem cells prepared from bone marrow, comprising treating the stem cells with gp96 molecules.

39. The method of claim 38, wherein the gp96 molecules are in combination with growth factors.

40. The method of claim 39, wherein the growth factor is GM-CSF.

41. The method of claim 38, wherein the gp96 molecules are unloaded gp96 molecules.

42. A population of APCs prepared a method of any of claims 1, 8, 34 or 38.

43. A therapeutic composition comprising APCs according to claim 42 and a therapeutically acceptable carrier.

44. A kit comprising gp96 molecules and reagents necessary for performing a method as in any of claims 1, 8, 34 or 38.
